(19) United States
(12) Patent Application Publication
(10) Pub. No.: US 2010/0331204 A1
(43) Pub. Date: Dec. 30, 2010

(54) METHODS AND SYSTEMS FOR ENRICHMENT OF TARGET GENOMIC SEQUENCES

(76) Inventors: Jeff Jeddeloh, Verona, WI (US); Todd Richmond, Madison, WI (US); Matthew Rodesch, Stoughton, WI (US); Daniel Gerhardt, Madison, WI (US); Paul Marrione, Middletown, WI (US); Thomas Albert, Verona, WI (US)

Correspondence Address: ROCHE DIAGNOSTICS OPERATIONS INC., 9115 Hague Road, Indianapolis, IN, 46250-0457, US

(21) Appl. No.: 12/695,447

(22) Filed: Jan. 28, 2010

Related U.S. Application Data
(60) Provisional application No. 61/152,287, filed on Feb. 13, 2009.

(51) Int. Cl. C40B 30/04; C07H 21/02; C07H 21/04

(52) U.S. Cl. 506/9; 536/23.1; 536/23.6

(57) ABSTRACT

The present invention provides methods and systems for targeted nucleic acid sequence enrichment in a sample. In particular, the present invention provides for enriching for targeted nucleic acid sequences during hybridizations in hybridization assays by first depleting non-target nucleic acid sequences.

### RELATED APPLICATIONS

This application claims priority to U.S. Provisional Application No. 61/152,287 filed Feb. 13, 2009.

### FIELD OF THE INVENTION

The present invention provides methods and systems for targeted genomic sequence enrichment. In particular, the present invention provides for enriching for targeted nucleic acid sequences during hybridizations in hybridization assays by depleting non-target nucleic acid sequences in a target genome.

### BACKGROUND OF THE INVENTION

The advent of nucleic acid microarray technology makes it possible to build an array of millions of nucleic acid sequences in a very small area, for example on a microscope slide (e.g., U.S. Pat. Nos. 6,375,903 and 5,143,854). Initially, such arrays were created by spotting pre-synthesized DNA sequences onto slides. However, the construction of maskless array synthesizers (MAS) as described in U.S. Pat. No. 6,375,903 now allows for the in situ synthesis of oligonucleotide sequences directly on the slide itself.

Using a MAS instrument, the selection of oligonucleotide sequences to be constructed on the microarray is under software control such that it is now possible to create individually customized arrays based on the particular needs of an investigator. In general, MAS-based oligonucleotide microarray synthesis technology allows for the parallel synthesis of millions of unique oligonucleotide features in a very small area of a standard microscope slide. With the availability of the entire genomes of hundreds of organisms, for which a reference sequence has generally been deposited into a public database, microarrays have been used to perform sequence analysis on nucleic acids isolated from a myriad of organisms.

Nucleic acid microarray technology has been applied to many areas of research and diagnostics, such as gene expression and discovery, mutation detection, allelic and evolutionary sequence comparison, genome mapping, drug discovery, and more. Many applications require searching for genetic variants and mutations across the entire human genome that underlies human diseases. In the case of complex diseases, these searches generally result in a single nucleotide polymorphism (SNP) or set of SNPs associated with diseases and/or disease risk. Identifying such SNPs has proved to be an arduous and frequently fruitless task because resequencing large regions of genomic DNA, usually greater than 100 kilobases (Kb), from affected individuals or tissue samples is required to find a single base change or to identify all sequence variants. Other applications involve the identification of gains and losses of chromosomal sequences which may also be associated with cancer, such as lymphoma (Martinez-Climent J A et al., 2003, Blood 101:3109-3117), gastric cancer (Weiss M M et al., 2004, Cell. Oncol. 26:307-317), breast cancer (Callagy G et al., 2005, J. Path. 205: 388-396) and prostate cancer (Paris, P L et al., 2004, Hum. Mol. Gen. 13:1303-1313). As such, microarray technology is a tremendously useful tool for scientific investigators and clinicians in their understanding of diseases and therapeutic regimen efficacy in treating diseases.

The genome is typically too complex to be studied as a whole, and techniques must be used to reduce the complexity of the genome. To address this problem, one solution is to reduce certain types of abundant sequences from a DNA sample, as found in U.S. Pat. No. 6,013,440. Alternatives employ methods and compositions for enriching genomic sequences as described, for example, in Albert et al. (2007, Nat. Meth., 4:903-5), Okou et al. (2007, Nat. Meth. 4:907-9), Olson M. (2007, Nat. Meth. 4:891-892), Hodges et al. (2007, Nat. Genet. 39:1522-1527) and as found in U.S. patent application Ser. Nos. 11/638,004, 11/970,949, and 61/032,594. Albert et al. disclose an alternative that is both cost-effective and rapid in effectively reducing the complexity of a genomic sample in a user defined way to allow for further processing and analysis. Lovett et al. (1991, Proc. Natl. Acad. Sci. 88:9628-9632) also describes a method for genomic selection using bacterial artificial chromosomes (BACs). Reducing the complexity of a genome by practicing target sequence enrichment followed by sequencing is far superior to measuring hybridization events alone. Hybridization events allow the hybridization of any species in a microarray or in solution; both target sequences and non-target sequences alike. By practicing complexity reduction and sequence enrichment, an investigator increases the on-target sequences captured (e.g., those sequences that are the focus of the assay) while decreasing the amount of non-target sequences captured (e.g., those not the focus of the assay).

However, an issue associated with any hybridization assay is the event of cross capture of non-target (e.g. repetitive) nucleic acid sequences, also known as secondary capture, of non-target nucleic acid sequences on the array or in solution during hybridization of the target nucleic acids. Secondary capture decreases the efficiency of complexity reduction in hybridization assays, in effect potentially swamping out the desired target capture by non-target capture leading to decreased target capture efficiency. Current methods suppress secondary capture by the addition of genomic blocker DNA, such as C₀t-1 DNA, to a hybridization assay. It would be preferential if no additional DNA was added to an experiment, but current practices do not provide that option.

As such, what are needed are methods for dealing with secondary capture in a hybridization assay by alternative methods that do not include the addition of unwanted nucleic acids while at the same time increase the efficiency of target nucleic acid capture for investigative endeavors.

### SUMMARY OF THE INVENTION

The present invention provides methods and systems for targeted sequence enrichment. In particular, the present invention provides for enriching for targeted nucleic acid sequences during hybridizations in hybridization assays by depleting non-target nucleic acid sequences in a target genome.

Secondary capture reactions on a microarray format lead to decreased efficiency in capturing target nucleic acids. This decreased efficiency is seen in the percent of on-target reads resulting from a microarray assay, such that when secondary capture is not suppressed or bypassed, the amount of non-target nucleic acids captured increases and the target nucleic acids decrease. The present invention is summarized as methods, systems and compositions for dealing with secondary capture in a microarray assay. Certain illustrative embodiments of the invention are described below. The present invention is not limited to these embodiments.

Embodiments of the present invention comprise immobilized nucleic acid probes to capture target nucleic acid sequences from, for example, a genomic sample by hybridizing the sample to probes, or probe derived amplicons, on a solid support or in solution. In the embodiments where hybridization takes place on a solid support or substrate, it is contemplated that the present invention is not limited to the solid support used. Solid supports or substrates include, but are not limited to, microarray substrates such as a slide, chip, beads, tube, column, wells, plates, and the like.

Hybridization reactions as described herein comprise applying a sample to one or more supports upon which are immobilized either non-target sequence probes or target sequence probes, or both. In one embodiment, a two stage scenario is provided wherein a sample is applied and hybridized to non-target sequence probes immobilized on a first support, the sample is removed (e.g., removed sample is depleted of non-target sequences) and hybridized to target sequence probes immobilized on a second support. The hybridized target sequences are then preferably eluted non-selectively, thereby depleting the sample of non-target sequences and enriching the target nucleic acid sequences without the use of a secondary capture blocker DNA.

In another embodiment, a one stage scenario is provided wherein a sample is applied and hybridized to one support upon which are located separate populations of both non-target sequence probes and target sequence probes, wherein hybridization occurs simultaneously for both non-target and target nucleic acid sequences. The hybridized target sequences are then non-selectively eluted from separate locations, thereby depleting the sample of non-target sequences and enriching the target nucleic acid sequences simultaneously without the use of a secondary capture blocker DNA. In preferred embodiments, the number or amount of immobilized non-target sequence probes on a support equals or exceeds the number or amount of non-target sequences as found in a sample for hybridization.

In some embodiments, the present invention provides for the enrichment of targeted sequences and depletion of non-targeted sequences (e.g., repetitive sequences), in a solution based format. In one preferred embodiment the two stage scenario is adapted to solution hybridization by a method comprising the following steps:
a) generating a first set of hybridization probes in solution comprising sequences complementary to non-target nucleic acid sequences;
b) generating a second set of hybridization probes on a microarray comprising sequences complementary to target nucleic acid sequences;
c) combining the first set of probes with the sample to allow the first set of probes to hybridize in solution to non-target nucleic acids;
d) removing the hybridized first set of probes from the sample to form a first enriched solution comprising the target nucleic acid sequences;
e) combining the second set of probes on the microarray with the first enriched solution to allow the second set of probes to hybridize to target nucleic acids;
f) removing the hybridized second set of probes; and
g) eluting the target sequences from the hybridized second set of probes to form a second enriched solution comprising the target nucleic acid sequences.

In another variation of the two stage solution phase method described above, both first and second sets of hybridization probes are generated in solution in steps a) and b) and step e) is performed in solution rather than on a microarray.

At the end of the two stage solution phase method, the enriched solution comprising target nucleic acid sequences is ready for downstream applications such as DNA or RNA sequencing, comparative genomic hybridization (CGH), and DNA methylation studies. Non-limiting examples of non-target sequences that may be removed by the two stage solution phase methods include repetitive sequences in genomic DNA (e.g., Alu, THE-1, LINE-1 repeats, etc), high abundance transcripts in messenger RNA (mRNA) or the complementary DNA (cDNA) from those high abundance transcripts, and ribosomal RNA (rRNA) sequences. Removal of non-target sequences improves the detection of target sequences such as rare transcripts and regulatory RNA. By removing these abundant transcripts, the effective sensitivity to detect rare transcripts through sequencing technologies increases, and the cost decreases. This benefit for rare transcript detection can be gained through either the two step depletion followed by positive selection for specific rare transcripts, or a single step depletion of abundant transcripts, followed directly by sequencing of the remaining molecular population.

In the two stage solution phase method described above, a particularly preferred embodiment is to generate the probes for hybridization in step a) from a microarray of immobilized probes. This is accomplished by means of a polymerase chain reaction on the immobilized probes to generate them in solution. Once in solution, the hybridization probes are further amplified and labelled by an asymmetric polymerase chain reaction using a 5′-biotinylated primer in excess over 3′-primer. After hybridization with sample in solution, the biotin-labelled probes are separated from unhybridized nucleic acid sequences using a streptavidin solid phase. The hybridized target sequences are finally eluted from the biotin labelled probes on the streptavidin solid phase.

Further embodiments of the present invention comprise immobilized nucleic acid probes to capture target nucleic acid sequences from, for example, a genomic sample by hybridizing the sample to probes, or probe derived amplicons, on a solid support or in solution, wherein the target nucleic acid is affixed with adapter linkers on one or both of the 5′ and 3′ ends of a fragmented nucleic acid sample, adapter linkers being useful for ligation mediated polymerase chain reaction (LM-PCR) methods and for sequencing applications. The captured target nucleic acids are preferably washed and non-selectively eluted off of the target sequence hybridization probes.

Genomic samples are used herein for descriptive purposes, but it is understood that other non-genomic samples could be subjected to the same procedures as the present invention provides for the depletion of non-target sequence capture in conjunction with any nucleic acid target regardless of origin. Increases in efficiency of target enrichment provided by the present invention offer investigators superior tools for use in research and therapeutics associated with disease and disease states such as cancers (Durkin et al., 2008, Proc. Natl. Acad. Sci. 105:246-251; Natrajan et al., 2007, Genes, Chr. And Cancer 46:607-615; Kim et al., 2006, Cell 125:1269-1281; Stallings et al., 2006 Can. Res. 66:3673-3680), genetic disorders (Balciuniene et al., Am. J. Hum. Genet. In press), mental diseases (Walsh et al., 2008, Science 320:539-543; Roohi et al., 2008, J. Med. Genet. Epub 18 Mar. 2008; Sharp et al., 2008, Nat. Genet. 40:322-328; Kumar et al., 2008, Hum. Mol. Genet. 17:628-638) and evolutionary and basic research (Lee et al., 2008, Hum. Mol. Gen. 17:1127-1136; Jones et al., 2007, BMC Genomics 8:402; Egan et al., 2007, Nat. Genet. 39:1384-1389; Levy et al., 2007, PLoS Biol. 5:e254; Ballif et al., 2007, Nat. Genet. 39:1071-1073; Scherer et al., 2007, Nat. Genet. S7-S15; Feuk et al., 2006, Nat. Rev. Genet. 7:85-97), to name a few.

The present invention provides methods of isolating and reducing the genetic complexity of a plurality of nucleic acid molecules, the method comprising the steps of exposing fragmented, denatured nucleic acid molecules of said population to the same or multiple, different oligonucleotide probes that are bound on a solid support under hybridizing conditions to capture nucleic acid molecules that specifically hybridize to said probes, or exposing fragmented, denatured nucleic acid molecules of said population to the same or multiple, different oligonucleotide probes under hybridizing conditions followed by binding the complexes of hybridized molecules to a solid support to capture nucleic acid molecules that specifically hybridize to said probes, wherein in both cases said fragmented, denatured nucleic acid molecules have an average size of about 100 to about 1000 nucleotide residues, preferably about 250 to about 800 nucleotide residues and most preferably about 400 to about 600 nucleotide residues, separating unbound and non-specifically hybridized nucleic acids from the captured molecules, non-selectively eluting the captured molecules, and optionally repeating the aforementioned processes for at least one further cycle with the eluted captured molecules and/or sequencing the enriched target nucleic acids.

In some embodiments, the target nucleic acid molecules are selected from an animal, a plant or a microorganism. If only limited samples of nucleic are available, the nucleic acids may be amplified, for example by whole genome amplification, prior to practicing the methods of the present invention. Prior amplification may be necessary for performing the inventive method(s), for example, for forensic purposes (e.g. in forensic medicine for genetic identity purposes).

In some embodiments, the population of target nucleic acid molecules is a population of genomic DNA molecules. In such embodiments, probes are selected from one or a plurality of sequences that, for example, define one or a plurality of exons, introns or regulatory sequences from a plurality of genetic loci, or a plurality of probes that define the complete sequence of at least one single genetic locus, said locus having a size of at least 100 kb, preferably at least 1 Mb, or at least one of the sizes as specified above, one or a plurality of probes that define single nucleotide polymorphisms (SNPs), or a plurality of probes that define an array, for example a tiling array designed to capture the complete sequence of at least one complete chromosome.

In some embodiments, the present invention comprises the step of ligating adapter molecules to one or both ends, preferably both ends, of the nucleic acid molecules prior to or after exposing fragmented nucleic samples to the probes for hybridization. In some embodiments, methods of the present invention further comprise the amplifying of the target nucleic acid molecules with at least one primer, said primer comprising a sequence which specifically hybridizes to the sequence of said adapter molecule(s). In some embodiments, the adapter molecules are self-complementary, non-complementary, or are Y-adapters (e.g., oligonucleotides that, once annealed, comprise a complementary end and a non-complementary end, the complementary end of which is annealed to fragmented nucleic acid samples). In some embodiments, the amplified target nucleic acid sequences may be sequenced, hybridized to a resequencing or SNP-calling array and the sequence or genotypes may be further analyzed.

In some embodiments, the present invention provides a complexity reduction method for target nucleic acid sequences in a genomic sample, such as exons or variants, preferably SNP sites. This can be accomplished by synthesizing one or more genomic probes specific for a region of the genome to capture complementary target nucleic acid sequences contained in a complex genomic sample. The enrichment methods comprise the inclusion of hybridization probes for targeting repetitive sequences in a particular genome.

In some embodiments, the present invention further comprises determining the nucleic acid sequence of the enriched and eluted target molecules, in particular by means of performing sequencing reactions.

In some embodiments, the present invention is directed to a kit comprising compositions and reagents for performing a method according to the present invention. Such a kit may comprise, but is not limited to, a double stranded adapter molecule, one or more solid supports comprising a plurality of hybridization probes for any particular microarray application (e.g., comparative genomic hybridization, expression, chromatin immunoprecipitation, comparative genomic sequencing, etc.), wherein said probes comprise sequences corresponding to both non-target sequences and target sequences as found in a genome on one or more of the solid supports. In some embodiments, a kit comprises two different double stranded adapter molecules. A kit may further comprise at least one or more other components selected from DNA polymerase, T4 polynucleotide kinase, T4 DNA ligase, hybridization solution(s), wash solution(s), and/or elution solution(s).

### DEFINITIONS

As used herein, the term “sample” is used .in its broadest sense. In one sense, it is meant to include a specimen or culture obtained from any source, preferentially a biological source, including either eukaryotic or prokaryotic. Biological samples may be obtained from animals (including humans) and encompass fluids, solids, and tissues. Biological samples include blood products, such as plasma, serum and the like. A sample from a non-human animal includes, but is not limited to, a biological sample from vertebrates such as rodents, non-human primates, ovines, bovines, ruminants, lagomorphs, porcines, caprines, equines, canines, felines, ayes, etc. Further, a sample as used herein includes biological samples from plants, for example a sample derived from any organism as found in the kingdom Plantae (e.g., monocot, dicot, etc.). A sample can also be from fungi, algae, bacteria, and the like. It is contemplated that the present invention is not limited to the origin of the sample. A sample as used herein is typically , a “sample of nucleic acids” or a “nucleic acid sample”, or a “target nucleic acid sample”, or a “target sample” comprising nucleic acids (e.g., DNA, RNA. cDNA, mRNA, tRNA, miRNA, rRNA, etc.) from any source. As such, a nucleic acid sample used in methods and systems of the present invention is a nucleic acid sample derived from any organism, either eukaryotic or prokaryotic.

For purposes of this invention, “target” or “target sequence” means a particular nucleic acid sequence of interest for investigation, isolation, amplification or other processes, and is defined to include either the single stranded sequence, the double stranded sequence, or sequences complementary thereto. For purposes of this invention,“non-target” or “non-target sequence” means nucleic acid sequences that are not of interest for these purposes, and is defined to include either the single stranded sequence, the double stranded sequence or sequences complementary thereto.

The pre-selected probes determine the range of targeted or non-targeted nucleic acid sequences. Thus, the “target” is sought to be sorted out from other nucleic acid sequences. A “segment” is defined as a region of nucleic acid within the target sequence, as is a “fragment” or a “portion” of a nucleic acid sequence. As such, “on-target reads” are the percentage or number of target nucleic acids that are sequenced and found to be the sequences desired by an investigator. “Repetitive nucleic acid sequences” are those sequences in a genome that are repetitive in nature and are known to contribute to secondary capture thereby affecting the efficiency of capture of target nucleic acid sequences.

As used herein, the term “isolate” when used in relation to a nucleic acid, as in “isolating a nucleic acid” refers to a nucleic acid sequence that is identified and separated from at least one component or contaminant with which it is ordinarily associated in its natural source. Isolated nucleic acid is in a form or setting that is different from that in which it is found in nature. In contrast, non-isolated nucleic acids are nucleic acids such as DNA and RNA found in the state they exist in nature. The isolated nucleic acid, oligonucleotide, or polynucleotide may be present in single-stranded or double-stranded form.

As used herein, the term “oligonucleotide,” refers to a short length of polynucleotide chain, preferably single-stranded. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer.” Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the term “hybridization” is used in reference to the pairing of complementary nucleic acids. Hybridization and the strength of hybridization (e.g., the strength of the association between the nucleic acids) is affected by such factors as the degree of complementarity between the nucleic acids, stringency of the conditions involved, the melting temperature (Tₘ) of the formed hybrid, and the G:C ratio of the nucleic acids. While the invention is not limited to a particular set of hybridization conditions, stringent hybridization conditions are preferably employed. Stringent hybridization conditions are sequence dependent and differ with varying environmental parameters (e.g., salt concentrations, presence of organics, etc.). Generally, “stringent” conditions are selected to be about 50° C. to about 20° C. lower than the Tₘ for the specific nucleic acid sequence at a defined ionic strength and pH. Preferably, stringent conditions are about 5° C. to 10° C. lower than the thermal melting point for a specific nucleic acid bound to a complementary nucleic acid. The Tₘ is the temperature (under defined ionic strength and pH) at which 50% of a nucleic acid (e.g., target nucleic acid) hybridizes to a perfectly matched probe.

“Stringent conditions” or “high stringency conditions,” for example, can be hybridization in 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 mg/ml), 0.1% SDS, and 10% dextran sulfate at 42° C., with washes at 42° C. in 0.2% SSC (sodium chloride/sodium citrate) and 50% formamide at 55° C., followed by a wash with 0.1×SSC containing EDTA at 55° C. By way of example, but not limitation, it is contemplated that buffers containing 35% formamide, 5×SSC, and 0.1% (w/v) sodium dodecyl sulfate (SDS) are suitable for hybridizing under moderately non-stringent conditions at 45° C. for 16-72 hours.

Furthermore, it is envisioned that the formamide concentration may be suitably adjusted between a range of 20-45% depending on the probe length and the level of stringency desired. Additional examples of hybridization conditions are provided in several sources, including Molecular Cloning: A Laboratory Manual, Eds. Sambrook et al., Cold Spring Harbour Press (incorporated herein by reference in its entirety).

Similarly, “stringent” wash conditions are ordinarily determined empirically for hybridization of a target to a probe, or in the present invention, a probe derived amplicon. The amplicon/target are hybridized (for example, under stringent hybridization conditions) and then washed with buffers containing successively lower concentrations of salts, or higher concentrations of detergents, or at increasing temperatures until the signal-to-noise ratio for specific to non-specific hybridization is high enough to facilitate detection of specific hybridization. Stringent temperature conditions will usually include temperatures in excess of about 30° C., more usually in excess of about 37° C., and occasionally in excess of about 45° C. Stringent salt conditions will ordinarily be less than about 1000 mM, usually less than about 500 mM, more usually less than about 150 mM (Wetmur et al., 1966, J. Mol. Biol., 31:349-370; Wetmur, 1991, Critical Reviews in Biochemistry and Molecular Biology, 26:227-259, incorporated by reference herein in their entireties).

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (e.g., in the presence of nucleotides and an inducing agent such as DNA polymerase and at a suitable temperature and pH). The primer is preferably single stranded for maximum efficiency in amplification. Preferably, the primer is an oligodeoxyribonucleotide. The primer must be sufficiently long to prime the synthesis of extension products in the presence of the inducing agent. The primer may be labelled with one member of a specific-binding pair such as a biotin for subsequent capture on a streptavidin support or a hapten (e.g. digoxigenin) for subsequent capture on a anti-hapten antibody support. The exact lengths of the primers will depend on many factors, including temperature, source of primer and the use of the method.

As used herein, the term “probe” refers to an oligonucleotide (e.g., a sequence of nucleotides), whether occurring naturally as in a purified restriction digest or produced synthetically, recombinantly or by PCR amplification, that is capable of hybridizing to at least a portion of another oligonucleotide of interest, for example target nucleic acid sequences. A probe may be single-stranded or double-stranded. Probes are useful in the detection, identification and isolation of particular gene sequences. A probe as used herein may be affixed to a microarray substrate, either by in situ synthesis using MAS or by any other method known to a skilled artisan, for subsequent hybridization to a target nucleic acid. Alternatively, a probe may be dissolved in a hybridization media for solution phase embodiments.

As used herein, the term “adapter” (or “adaptor”) is a double stranded oligonucleotide of defined (or known) sequence which is affixed to one or both ends of sample DNA molecules. Sample DNA molecules may be fragmented or not before their addition. In the case where adapters are added to both ends of the sample DNA molecule, the adapters may be the same (i.e homologous sequence on both ends) or different (i.e heterologous sequences at each end). For the purposes of ligation-mediated polymerase chain reaction (LM-PCR), the terms “adapter” and “linker” are used interchangeably. The two strands of the adapter may be self-complementary, non-complementary or partially complementary (e.g. Y-shaped). Adapters typically range from 12 nucleotide residues to 100 nucleotide residues, preferably from 18 nucleotide residues to 100 nucleotide residues, most preferably from 20 to 44 nucleotide residues.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

### FIGURES

FIGS. 1A-B exemplifies a two stage target sequence enrichment method on commercial microarrays and adapters for sequencing. In step 1, a DNA sample is fragmented and converted to a 454 Life Sciences sequencing library with adapters attached to the 3′ and 5′ termini. The library is then amplified by PCR in step 2. Then in step 3 the adaptor-ligated DNA sample is hybridized to a first microarray consisting of forward and reverse probes corresponding to repetitive DNA elements. The first microarray is removed from the solution, along with hybridized repetitive DNA, resulting in a sample depleted in repetitive DNA (step 4). Next, target regions are identified and a second microarray is designed to capture these regions of interest. The library is hybridized to the second microarray for up to 3 days in step 5. The second microarray is washed in step 6 then targeted DNA is eluted non-selectively from the microarray in step 7. The eluted target DNA is amplified in step 8, and sequenced in step 9.

FIG. 2 exemplifies one embodiment of the present invention for a generic two stage target sequence enrichment method. A) A microarray comprising repetitive probe sequences is hybridized to a fragmented linker-adapted genomic library comprising both repetitive and target genomic sequences using a gasket slide (B) to create a hybridization chamber. C) The solution from the first hybridization is hybridized to a second microarray comprising target probe sequences under an additional gasket slide to create a hybridization chamber (D). The enriched target genomic sequences are eluted thereby providing a genomic library enriched for target sequences and depleted of unwanted repetitive sequences.

FIG. 3 exemplifies another embodiment of the present invention for a one stage target sequence enrichment method. A) A microarray comprising both repetitive probe sequences and target probe sequences are found on a microarray and a fragmented linker adapted genomic library is applied to simultaneously to both and hybridization in a hybridization chamber created by application of a mixer apparatus (B) is allowed to occur. C) Enriched target genomic sequences are eluted from the target probe array only thereby providing a genomic library enriched for target sequences and depleted of unwanted repetitive sequences.

FIGS. 4A and 4B exemplify covers used for repeat subtraction on NimbleGen microarray substrates. Both covers are shown first in a flat orientation and second in an sideon orientation. In the sideon orientations, the layers of materials comprising the covers are indicated. FIG. 4A shows the dimensions of a HX3 cover which divides the hybridization chamber into three equal sections with 2 ports each for a total of 6 ports. FIG. 4B shows the dimensions of an HX1 cover which encompasses the hybridization in a single section with 2 ports.

FIG. 5 exemplifies solution sequence capture probe pool generation. Probe pools are generated by amplifying probes from an array (In situ) with 30 cycles of PCR. One strand of the DNA is selected for by asymmetric PCR, producing multiple copies of single stranded DNA; this is done for the forward and reverse strand of the target DNA. The probes are purified and quantified before being used in repeat subtraction (Patent WO200905039).

FIG. 6 exemplifies a solution phase repeat capture experiment Forward and reverse probes are added to DNA sample, which will hybridize to repetitive DNA elements. The probes are removed from the solution, along with repetitive DNA, resulting in a sample depleted in repeats and ready for downstream applications like Sequence Capture direct sequencing, comparative genomic hybridization (CGH) and methylation studies.

FIG. 7 exemplifies a workflow for preparing bacterial artificial chromosome (BAC) sequences within a fingerprint contiguous region (FPC ctg138) for probe design.

### DETAILED DESCRIPTION OF THE INVENTION

Secondary capture in microarray assays comprises the hybridization based interaction of sequences not represented in the microarray target probe capture design (e.g., Alu, THE-1, LINE-1 repeats, etc.). One type of secondary capture, for example, is found between non-hybridized sample DNA and the target DNA that is hybridized to a probe (“sequence mediated secondary capture”). For example, in secondary capture a probe specifically hybridizes to its target, but that target has some non-probe sequences (e.g., Alu, THE-1, LINE-1 repeats, etc.) that also hybridize to non-cis copies. One consequence of secondary capture is the enrichment of specific subsets of repeat elements within a target sample (e.g., non-target or repetitive sequences), leading to poor overall enrichment of the target region. In essence, the desired target sequence to be enriched by capture on the microarray is swamped out by the co-enrichment of unwanted types of local sequence repeats.

Competitive, or suppression, hybridization to block secondary capture involves blocking the capture of a potentially strong repetitive DNA signal which can be obtained when using a complex DNA. For example, the DNA is denatured and allowed to re-anneal in the presence of total genomic. DNA in solution, or preferably a fraction that is enriched for highly repetitive DNA sequences. In either case, the highly repetitive DNA within the target DNA is present in large excess over the repetitive elements in the probe (since the arrays are most often produced with as little repeat as possible). As a result, such sequences will readily associate with complementary strands of the repetitive sequences within the target, adding massive excess of exogenous copies of the same types of repeats thereby effectively blocking their hybridization to target sequences. As such, blocking agents are typically used during hybridization reactions.

Recently, it was demonstrated that enrichment of target sequences in, for example, plant species is more efficient when species specific blocking DNA (e.g., C₀t-1) is included during the hybridization reaction in a microarray assay. It is contemplated that this is due to the supression of secondary capture. However, production of sufficient quantities of plant derived C₀t-1 DNA, for example from corn, is problematic in terms of time and resources.

As such, alternative methods for bypassing the use of a blocker in enrichment processes and methods was investigated. In one such method, non-redundant statistically derived repeats (SDRs) from the MAGI Cereal Repeat Database version 3.1 and sequences from the TIGR Maize Repeat Database were utilized to design an all repeat (maize) microarray. The design was verified by NCBI's Megablast to compare a collection of 454 Life Sciences derived sequencing reads from maize B73 to the database of repeat sequences used to construct the array. A total of over 271,000 reads (>102 Mbp) was used in the comparison. Analysis demonstrated that 75% of the total sequence had 90% or higher identity to the maize repeat sequences. This is in close agreement with the established repeat burden in the maize genome, and approximately identical to the percent of input reads that were computationally masked. As such, it is contemplated that the all repeat design accurately reflects the repeat content of the maize genome as an example system. Consequently, hybridization reactions were designed to utilize the repeat design for depletion of repeat regions in a maize genome prior to, or concurrently with, hybridization of target nucleic acid sequences to target sequence probes.

It is further contemplated that the methods for depletion of repetitive sequences from a genome as described herein is amenable to any hybridization assay, either on a solid phase such as a microarray slide or in solution.

Existing protocols for capturing target plant sequences in a genomic sample call for investigators to dry down plant genomic DNA with, for example, 100 μg of C₀t-1 DNA, followed by reconstitution in a hybridization buffer and hybridization sample in a hybridization assay. The current exemplary protocol makes the addition of a blocker surprisingly unnecessary while still maintaining selective target sequence capture.

As described herein, methods, systems and compositions of the present invention provide for the depletion of non-target or repetitive sequences in hybridization assays thereby increasing the capture of target sequences in a target genome. Certain illustrative embodiments of the invention are described below. The present invention is not limited to these embodiments.

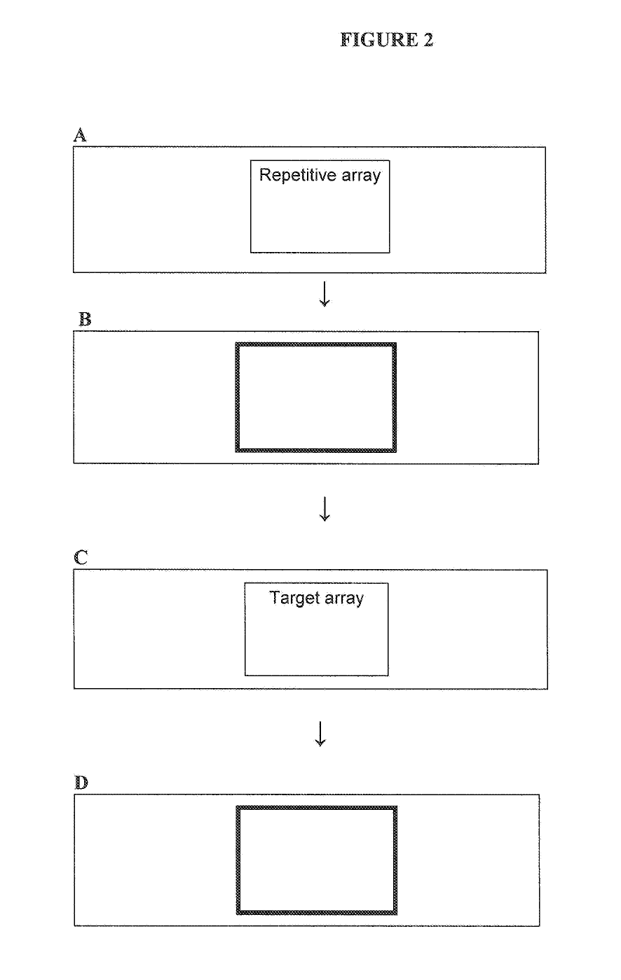

In one embodiment of the present invention, two microarrays are designed, for example using maskless array synthesis; one array comprises probe sequences that are repetitive in nature for binding to repetitive sequences in the plant genome while the other array is designed to contain probe sequences for hybridizing to target sequences (FIG. 2).

A library of plant genomic sequences is created by attaching adapter, or linker molecules, to one or both ends of fragmented genomic DNA such as that created using a GS FLX Titanium Library Preparation Kit (454 Life Sciences, Branford, Conn.). In an exemplary protocol, the following components are added to a 1.5 ml tube and heated for 10 minutes at 95° C.: 65 μl Hybridization component A, 26.6 μl Formamide, 2.0 μl Tween-20, 1 μl of Enhancing oligos A and B (454 Titanium kit), 500 ng of Linker adapted DNA generated using 454 Titanium Library prep kit and water to a final volume of 125 μl.

A gasketed slide (FIG. 2B) (for example as provided by SciGene Corporation, Sunnyvale, Calif.) or a hybridization chamber (for example as provided by Grace Bio-Labs Corporation, Bend, Oreg.) (Repeat Subtraction figures) is placed on a Mai Tai® Hybridization System mixer assembly (SciGene Corporation). The DNA mixture is pipetted onto the gasket slide. A microarray comprising repetitive sequence probes (FIG. 2A) is inverted and placed face down on the gasket slide such that the probes are in contact with the heated sample. The top of the Mai Tai® mixer assembly is screwed down firmly and placed in a SciGene incubator for hybridization at 42° C. for 4 days on mix setting 15. Alternatively a hybridization chamber is affixed to the repeat array and the sample is loaded into this chamber. This is then put into the Mai Tai® mixer and placed in a SciGene incubator for hybridization at 42° C. for 4 days on mix setting 15. After hybridization, the mixer assembly is disassembled, the microarray slide is separated from the gasket array slide and the hybridization mixture is rescued from the slide. During the first hybridization with the repetitive probe microarray it is contemplated that the repetitive sequences as found in the linker adapted library are hybridized to the microarray leaving in solution target genomic sequences. The system described herein is for exemplary purposes only, and any system that allows for the creation of a hybridization chamber and subsequent rescue of a sample post hybridization is equally amenable for use with the present invention.

A second round of hybridizations occurs; however, instead of utilizing a repetitive probe microarray, a microarray with probes to target genomic sequences is utilized (FIG. 2C). For example, the solution that is rescued from gasket slide after removal of the repetitive array is heated for 5 min at 95° C. for 5 min. and placed on a gasket slide (FIG. 2D) upon which is placed the target probe microarray. The second hybridization reaction comprises target probe sequences hybridized to target genomic sequences as found in the genomic library. Target genomic linker adapted sequences are subsequently eluted from the target microarray with sodium hydroxide thereby providing enriched samples for sequencing without the use of an initial blocker DNA to block secondary capture of unwanted non-target repetitive genomic sequences.

In some embodiments, the repetitive sequence depleted hybridization mixture from the first hybridization is applied to a Qiagen MinElute column, for example, and bound DNA is eluted with water thereby separating the target genomic sequences from the hybridization reaction components. The purified target genomic sequences are applied to a sequence capture workflow for target enrichment, for example by following established protocols as found in NimbleGen Array User's Guide Sequence Capture Array Delivery (Roche NimbleGen, Inc., Madison, Wis.) and target genomic captured sequences and then eluted as described. In some embodiments, the target sequences as found in the solution after the first hybridization but prior to the second hybridization are amplified (for example, by LM-PCR) before hybridization with the target sequence probes. Regardless of the target hybridization method used, the captured target sequences are non-selectively eluted from the target capture array using, for example 400 l of 100 mM NaOH which removes not only specifically hybridized target sequences but also any non-specifically bound nucleic acids. The eluent is then separated from reaction components using, for example, a Qiagen MinEute column. The enriched and eluted target genomic regions are then applied to downstream applications in preparation for, for example, sequencing utilizing the 454 GS FLX Titanium system (454 Corporation).

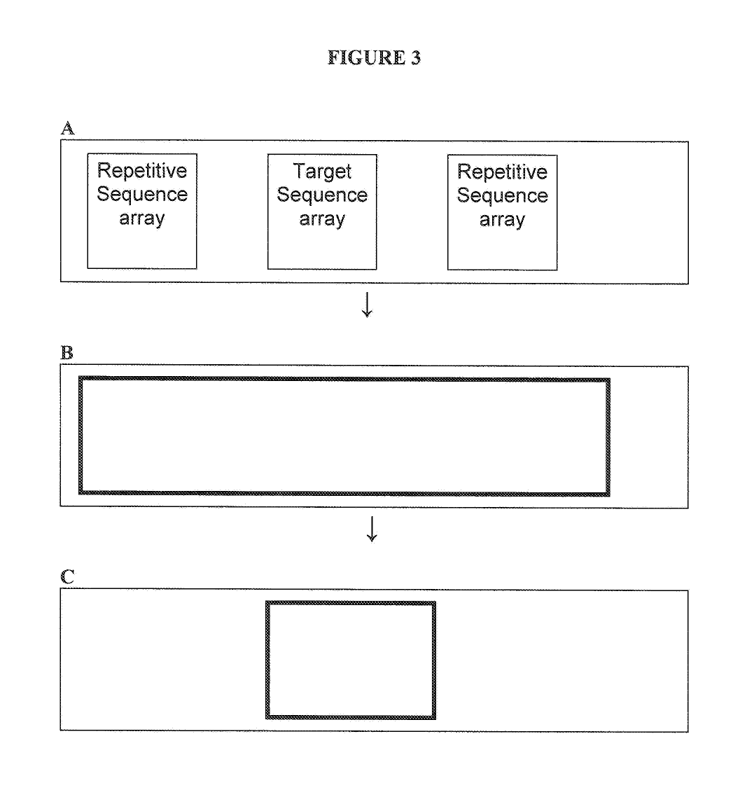

An alternative to a two array slide workflow is a one array slide workflow. For example, a microarray is designed as found in the FIX3 slide provided by Roche NimbleGen. Inc. comprising three separated arrays on one slide as exemplified in FIG. 3. An arrangement of one or both of the arrays on the ends of the slide contain repetitive probe sequences, whereas the middle array contains target probe sequences. A cover slip, for example as provided by BioMicro Corporation, is placed over all the arrays thereby creating a hybridization chamber and a hybridization mixture as described above is pipetted into the hybridization chamber. Mixing and hybridization is allowed to occur wherein fluid communication is maintained between all two or three of the array fields, for example as described in the NimbleGen Array User's Guide Sequence Capture Array Delivery. Target sequences are eluted following the protocol defined for the Elution Station (Roche NimbleGen, Inc.), wherein only those bound target sequences as hybridized on the middle array are non-selectively eluted from the microarray slide. As such, the unwanted repetitive sequences remain bound on the array whereas the enriched and eluted target genomic sequences are utilized in downstream sequencing applications.

In one embodiment of the present invention, hybridization probes are designed that will both capture repetitive sequences in a genome while concurrently capturing target sequences in a genome. In one embodiment, utilizing maskless array synthesis (or any other method for synthesizing probes on a support as the present invention is not limited to the microarray synthesis method or process), a support such as a microarray slide comprising two or more separate array fields is designed and probes are synthesized on the support in the array fields. At least one of the array fields is designed to comprise hybridization probes hybridizable to target nucleic acid sequences and at least one of the array fields is designed to comprise hybridization probes hybridizable to repetitive nucleic acid sequences of a genome (FIG. 3A). The present invention is not limited by the number of array fields on the support, indeed at least 2, at least 3, at least 4, at least 6, at least 12 fields are anticipated for use in methods of the present invention.

A sample comprising repetitive and target sequences is added to the array, typically under a cover slip device that allows for the formation of a hybridization chamber, for example as provided by placing a NimbleGen mixer apparatus (for example HX1 Mixer, Roche NimbleGen, Inc., Madison Wis.) over the microarray whereby an enclosed hybridization chamber is created between the slide and the mixer (FIG. 3B). Hybridization is allowed to occur between the probes and sample nucleic acids for a pre-determined time period, e.g., at least 1 day, at least 2 days, at least 3 days, at least 4 days. It is contemplated that during hybridization repetitive sequences will preferentially hybridize to the repetitive probe sequences, whereas the target sequences will preferentially hybridize to the target probe sequences. After hybridization, the coverslip (e.g., mixer) is removed and preferentially the support is washed one or more times to remove non-hybridized and/or weakly hybridized sequences. In preferred embodiments, the target nucleic acids hybridized to the target probes sequences are selectively eluted from the support (FIG. 3C), for example by utilizing a NimbleGen Elution System (Roche NimbleGen, Inc.) and not eluting the hybridized repetitive sequences. In some embodiments, the eluted target is sequenced, for example sequencing utilizing the 454 GS FLX Titanium system (454 Corporation).

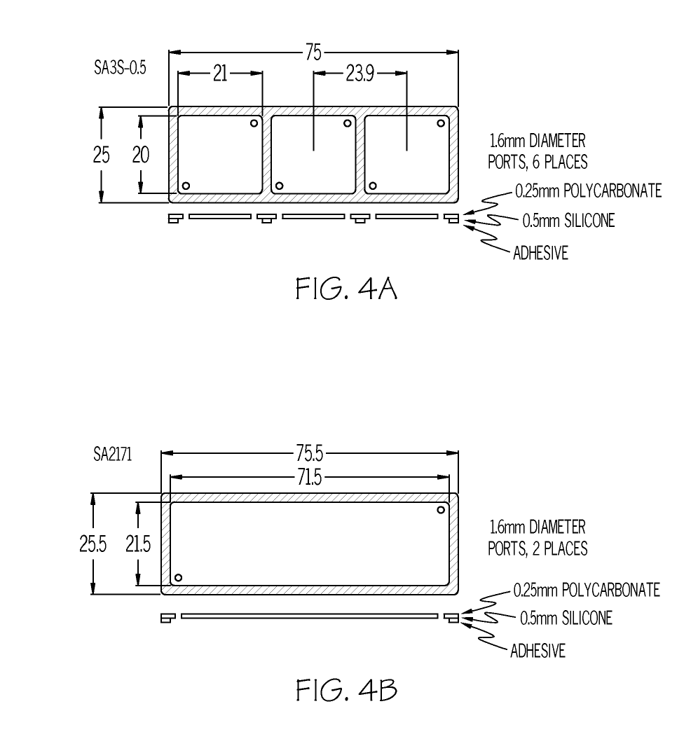

In one embodiment the repeat subtraction is done on a HX3 array or HX1 array available from Roche NimbleGen Inc. as shown in FIG. 4. This will allow for repeat subtraction from larger array formats.

In some embodiments, the present invention provides nucleic acid molecules comprising adaptors, for example ligation mediated or LM-PCR adapters, on one or both ends of the DNA molecules. In some embodiments, these adaptors as affixed to the ends of target, fragmented DNA allows for, for example, the amplification of genomic DNA prior to the enrichment, with enrichment of target sequences occuring from the amplified population. One exemplary method for adapter attachment is by making a sequencing library, for example, by using a library protocol wherein the enriched targets can be sequenced directly in a sequence analysis protocol from 454 Life Sciences (Branford, Conn.) using a GS FLX sequencer. However, the present invention is not limited by the method used for library generation and sequencing and the present example demonstrates only one possible embodiment of the present invention (e.g., a skilled artisan will recognize alternative methods equally amendable for use with the present invention).

In some embodiments of the present invention, a sample containing denatured (e.g., single-stranded) nucleic acid molecules, preferably genomic nucleic acid molecules, which can be fragmented molecules, is exposed under hybridizing conditions to a plurality of oligonucleotide probes on a microarray substrate.

In some embodiments of the present invention, a sample containing nucleic acid molecules, preferably genomic nucleic acid molecules, which can be fragmented molecules, are further modified to comprise adapter linker sequences on both the 5′ and 3′ ends of the fragmented DNA. The adapter sequences can either be self-complementary, non-complementary, or Y type adapters. The adapter sequences are utilized, for example, for ligation mediated amplification of the fragmented nucleic acids as well as for sequencing purposes. Adapter linked fragments are preferentially amplified via LM-PCR and are exposed under hybridizing conditions to a plurality of oligonucleotide probes on a microarray substrate.

It is contemplated that the present invention is not limited by the kind of microarray assay being performed, and indeed any assay where depletion of non-target regions is desired will benefit from practicing the methods and systems of the present invention. Assays include, but are not limited to, complexity reduction and sequence enrichment, comparative genomic hybridization, comparative genomic sequencing, expression, chromatin immunoprecipitation-chip (ChIP-chip), epigenetic, and the like.

In embodiments of the present invention, probes for capture of target nucleic acids are immobilized on a substrate by a variety of methods. In one embodiment, probes can be spotted onto slides (e.g., U.S. Pat. Nos. 6,375,903 and 5,143,854). In preferred embodiments, probes are synthesized in situ on a substrate by using maskless array synthesizers (MAS) as described in U.S. Pat. No. 6,375,903, 7,037,659, 7,083,975, 7,157,229 that allows for the in situ synthesis of oligonucleotide sequences directly on a slide.

In some embodiments, a solid support is a population of beads or particles. The beads may be packed, for example, into a column so that a target sample is loaded and passed through the column and hybridization of probe/target sample takes place in the column, followed by washing and elution of target sample sequences for reducing genetic complexity and enhancing target capture. In some embodiments, in order to enhance hybridization kinetics, hybridization takes place in an aqueous solution comprising multiple probes in suspension in an aqueous environment.

In embodiments of the present invention, the hybridization probes for use in microarray capture methods as described herein are printed or deposited on a solid support such as a microarray slide, chip, microwell, column, tube, beads or particles. The substrates may be, for example, glass, metal, ceramic, polymeric beads, etc. In preferred embodiments, the solid support is a microarray slide, wherein the probes are synthesized on the microarray slide using a maskless array synthesizer. The lengths of the multiple oligonucleotide probes may vary and are dependent on the experimental design and limited only by the possibility to synthesize such probes. In preferred embodiments, the average length of the population of multiple probes is about 20 to about 100 nucleotides, preferably about 40 to about 85 nucleotides, in particular about 45 to about 75 nucleotides. In embodiments of the present invention, hybridization probes correspond in sequence to at least one region of a genome and can be provided on a solid support in parallel using, for example, maskless array synthesis (MAS) technology.

The present invention is not limited to the type of sample for capture, and indeed it is contemplated that any sample used is equally applicable to the present invention including, but not limited to, genomic DNA or RNA sample, cDNA library or mRNA library. In some embodiments, nucleic acid sequences used herein are fragmented, wherein said fragments have an average size of about 100 to about 1000 nucleotide residues, preferably about 250 to about 800 nucleotide residues and most preferably about 400 to about 600 nucleotide residues.

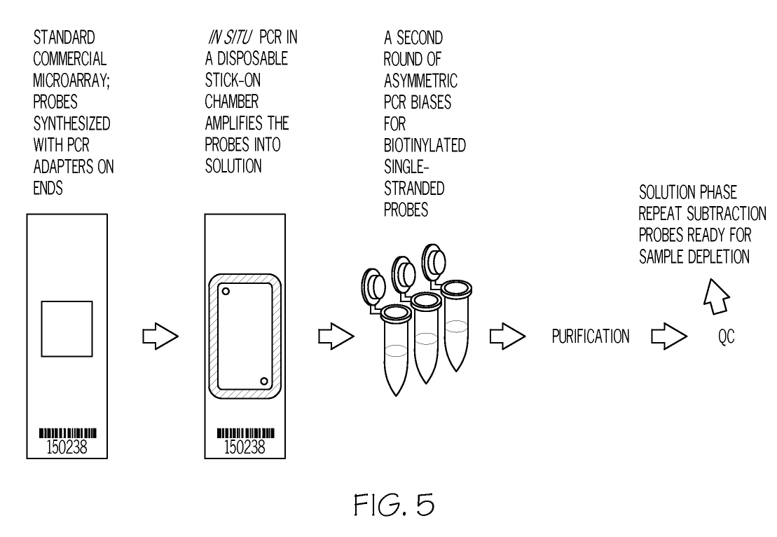

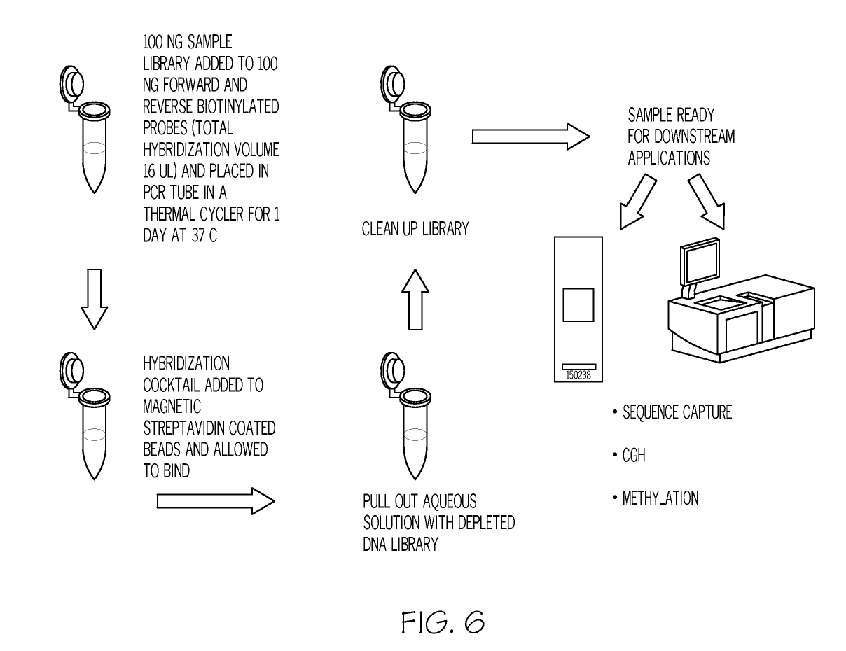

In another embodiment, the first stage of a two stage scenario for removing non-target sequences followed by isolation of target sequences is performed in solution as shown in FIGS. 5 and 6. Thus, repetitive sequence probes on a first solid support are first subjected to a polymerase chain reaction (PCR) in order to amplify the probes into solution (FIG. 5). The probes in solution are then subjected to a second round of asymmetric PCR with a 5′-biotinylated primer in order to obtain biotinylated single-strand probes. The biotinylated probes are then hybridized in solution to sample (FIG. 6). The first hydridization mixture is then exposed to streptavidin-coated solid support to remove the biotinylated hybridized non-target sequences. The sample now depleted of non-target sequences is then ready for the second stage of target sequence capture either on a solid support (e.g. microarray) or in solution. Alternatively, the depleted sample can be used for other downstream applications such as direct sequencing, comparative genomic hybridization (CGH) or methylation studies.

For the two stage solution phase embodiment, one skilled in the art will recognize that other specific binding partners may be substituted for the biotin and streptavidin pair, for example hapten labelled probes paired with anti-hapten antibody on a solid support. (e.g. digoxigen-labelled probes and anti-digoxigenin antibody).

In embodiments of the present invention, target nucleic acids are typically deoxyribonucleic acids or ribonucleic acids, and include products synthesized in vitro by converting one nucleic acid molecule type (e.g., DNA, RNA and cDNA) to another as well as synthetic molecules containing nucleotide analogues. Fragmented genomic DNA molecules are in particular molecules that are shorter than naturally occurring genomic nucleic acid molecules. A skilled person can produce molecules of random or non-random size from larger molecules by chemical, physical or enzymatic fragmentation or cleavage using well known protocols. For example, chemical fragmentation can employ ferrous metals (e.g., Fe-EDTA), physical methods can include sonication, hydrodynamic force or nebulization (e.g., see European patent application EP 0 552 290) and enzymatic protocols can employ nucleases and partial digestion reactions such as micrococcal nuclease (Mnase) or exo-nucleases (such as Exo1 or Ba131) or restriction endonucleases.

The population of nucleic acid molecules which may comprise the target nucleic acid sequences can vary from quite small to very large. In particular, the size(s) of the nucleic acid molecule(s) is/are at least about 100 bases, at least about 10 kilobases (kb), at least about 100 kb, at least about 1 megabase (Mb), at least about 100 Mb, especially a size between about 100 bases and about 10 kb, between about 10 kb and about 100 Mb, between about 100 kb and about 100 Mb, between about 1 Mb and about 100 Mb. In some embodiments, the nucleic acid molecules are genomic DNA, while in other embodiments the nucleic acid molecules are cDNA, or RNA species (e.g., tRNA, mRNA, miRNA). RNA or cDNA can be used to deplete abundant transcripts, such as ribosomal protein mRNAs or other highly expressed RNA species. By removing abundant molecules before sequencing, the sensitivity to detecting rare transcripts, such as regulatory RNAs, will be increased, and the cost of sequencing rare transcripts will be decreased.

In embodiments of the present invention, the nucleic acid molecules which may or may not comprise the target nucleic acid sequences may be selected from an animal, a plant or a microorganism. In some embodiments, if limited samples of nucleic acid molecules are available the nucleic acids are amplified (e.g., by whole genome amplification) prior to practicing the method of the present invention. For example, prior amplification may be necessary for performing embodiments of the present invention for forensic purposes (e.g., in forensic medicine, etc.).

In some embodiments, the population of nucleic acid molecules is a population of genomic DNA molecules. The hybridization probes and subsequent amplicons may comprise one or more sequences that target one or more (e.g., a plurality of) exons, introns or regulatory sequences from one ore more (e.g., a plurality of) genetic loci, the complete sequence of at least one single genetic locus, said locus having a size of at least 100 kb, preferably at least 1 Mb, or at least one of the sizes as specified above, sites known to contain SNPs, or sequences that define an array, in particular a tiling array, designed to capture the complete sequence of at least one complete chromosome. In some embodiments, only one hybridization probe sequence is utilized to capture a target sequence. Indeed, the present invention is not limited to the number of different probe sequences utilized to capture a target nucleic acid.

It is contemplated that target nucleic acid sequences are enriched from one or more samples that include nucleic acids from any source, in purified or unpurified form. The source need not contain a complete complement of genomic nucleic acid molecules from an organism. The sample, preferably from a biological source, includes, but is not limited to, isolates from individual patients, tissue samples, or cell culture. The target region can be one or more continuous blocks of several megabases, or several smaller contiguous or discontiguous regions, such as all of the exons from one or more chromosomes, or sites known to contain SNPs. For example, the one or more hybridization probes comprising one, or multiple different, sequence(s) and subsequent probe derived amplicons can support an array (e.g., non-tiling or tiling) designed to capture one or more complete chromosomes, parts of one or more chromosomes, one exon, all exons, all exons from one or more chromosomes, selected one or more exons, introns and exons for one or more genes, gene regulatory regions, and so on.

Alternatively, to increase the likelihood that desired non-unique or difficult-to-capture targets are enriched, the probes can be directed to sequences associated with (e.g., on the same fragment as, but separate from) the actual target sequence, in which case genomic fragments containing both the desired target and associated sequences will be captured and enriched. The associated sequences can be adjacent or spaced apart from the target sequences, but a skilled person will appreciate that the closer the two portions are to one another, the more likely it will be that genomic fragments will contain both portions.

In some embodiments of the present invention, the methods comprise the step of ligating adapter or linker molecules to one or both ends of fragmented nucleic acid molecules prior to denaturation and hybridization to the probes. In some embodiments of the present invention the methods further comprise amplifying said adapter modified nucleic acid molecules with at least one primer, said primer comprising a sequence which specifically hybridizes to the sequence of said adapter molecule(s). In some embodiments of the present invention, double-stranded adapters are provided at one or both ends of the fragmented nucleic acid molecules before sample denaturation and hybridization to the probes. In such embodiments, target nucleic acid molecules are amplified after elution to produce a pool of amplified products having further reduced complexity relative to the original sample. The target nucleic acid molecules can be amplified using, for example, non-specific Ligation Mediated-PCR (LM-PCR) through multiple rounds of amplification and the products can be further enriched, if required, by one or more rounds of selection against the microarray probes. The linkers or adapters are provided, for example, in an arbitrary size and with an arbitrary nucleic acid sequence according to what is desired for downstream analytical applications subsequent to the complexity reduction step. The adapter linkers can range between about 12 and about 100 base pairs, including a range between about 18 and 100 base pairs, and preferably between about 20 and 44 base pairs. In some embodiments, the linkers are self-complementary, non-complementary, or Y adapters.

Ligation of adapter molecules allows for a step of subsequent amplification of the captured molecules. Independent from whether ligation takes place prior to or after the capturing step, there exist several alternative embodiments. In one embodiment, one type of adapter molecule (e.g., adapter molecule A) is ligated that results in a population of fragments with identical terminal sequences at both ends of the fragment. As a consequence, it is sufficient to use only one primer in a potential subsequent amplification step. In an alternative embodiment, two types of adapter molecules A and B are used. This results in a population of enriched molecules composed of three different types: (i) fragments having one adapter (A) at one end and another adapter (B) at the other end, (ii) fragments having adapters A at both ends, and (iii) fragments having adapters B at both ends. The generation of enriched molecules with adapters is of outstanding advantage, if amplification and sequencing is to be performed, for example using the 454 Life Sciences Corporation GS20 and GS FLX instrument (e.g., see GS20 Library Prep Manual, December 2006, and WO 2004/070007; incorporated herein by reference in their entireties).

In preferred embodiments, the methods of the present invention are utilized in depleting repeat regions in plant genomic regions in a hybridization assay. It is contemplated that the present invention is not limited to any particular plant species. Examples of plant species utilized with the present invention include, but are not limited to, economically and/or research relevant plant species such as corn, soybean, sorghum, wheat, rice, barley, sugarcane, vegetable crops, fruit crops, forage crops, grasses, broadleaf plants and any other dicot and/or monocot plants.

In other embodiments, the methods of the present invention are utilized in non-plant genomes with very high repeat content such as fish and salamanders.

In some embodiments, the present invention comprises a kit comprising reagents and materials for performing methods according to the present invention. Such a kit may include one or substrates upon which is immobilized a plurality of hybridization probes specific to one or more target nucleic acid sequences from one or more target genetic loci (e.g., specific to exons, introns, SNP sequences, etc.), a plurality of probes that define a tiling array designed to capture the complete sequence of at least one complete chromosome, hybridization probes specific to repetitive nucleic acid sequences in a target genome, amplification primers, reagents for performing polymerase chain reaction methods (e.g., salt solutions, polymerases, dNTPs, amplification buffers, etc.), reagents for performing ligation reactions (e.g., ligation adapters, T4 polynucleotide kinase, ligase, buffers, etc.), tubes, hybridization solutions, wash solutions, elution solutions, magnet(s), and tube holders. In some embodiments, a kit further comprises two or more different double stranded adapter molecules.

In some embodiments, a kit further comprises at least one or more compounds from a group consisting of DNA polymerase, T4 polynucleotide kinase, T4 DNA ligase, one or more array hybridization solutions, and/or one or more array wash solutions. In preferred embodiments, three wash solutions are included in a kit of the present invention, the wash solutions comprising SSC, DTT and optionally SDS. For example, kits of the present invention comprise Wash Buffer 1 (0.2% SSC, 0.2% (v/v) SDS, 0.1 mM DTT), Wash Buffer II (0.2% SSC, 0.1 mM, DTT) and/or Wash Buffer III (0.05% SSC, 0.1 mM DTT). In some embodiments, systems of the present invention further comprise a non-selective elution solution, for example—a solution containing sodium hydroxide.

### Examples

The following examples are illustrative of the invention and are not limiting in any way. to the practice of the invention:

### Example 1

### Array-Based Repeat Subtraction-Mediated Sequence Capture (RSSC) for Maize

### Repeat Array Design

A custom 720K NimbleGen microarray (081110_{—}Zea_{—}mays_repeats_cap) was synthesized three times per slide to contain maize repetitive elements in the MAGI Cereal Repeat Database (v3.1; http://magi.plantgenomics.iastate.edu/repeatdb.html) and the TIGR Maize Repeat Database (v4; http://maize.jcvi.org/repeat_db.shtml). The design may be ordered by request. There are 2.1M total probes on the array. Only the center subarray containing 720K probes was utilized in this study.

### Maize NimbleGen Capture Array Design

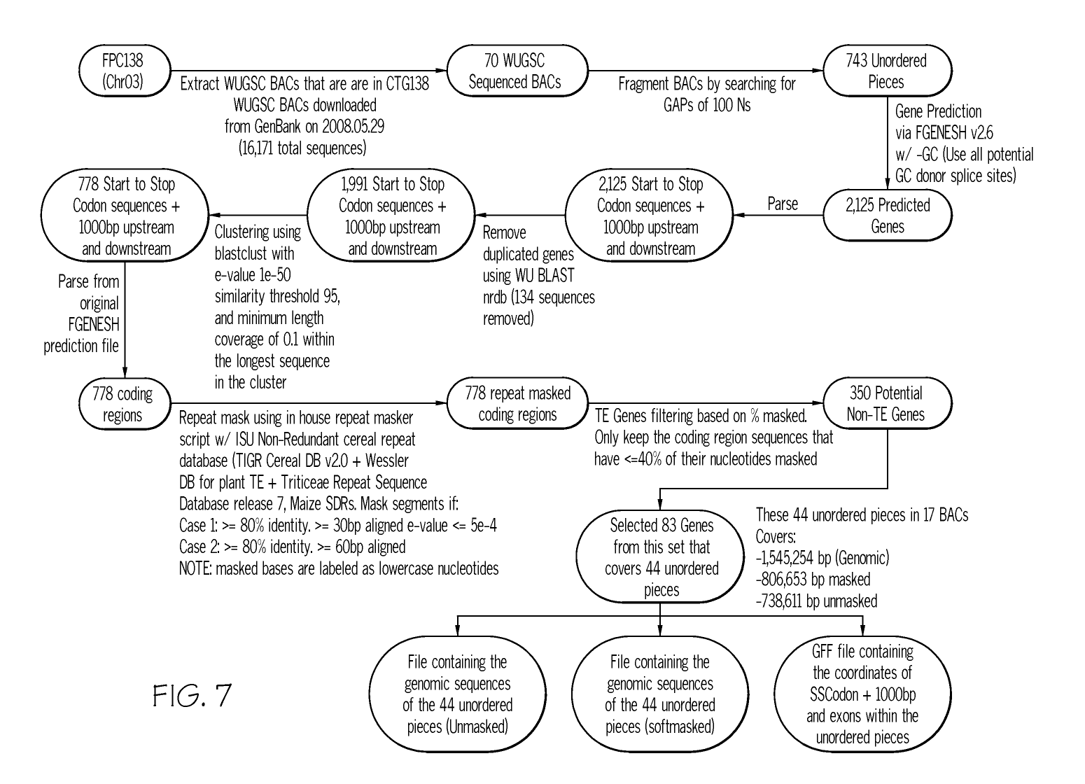

A large genomic region on a BAC fingerprint contig (FPC Ctg138, chr 3) was originally selected for targeting. Based on the physical map released prior to May 29, 2008, a total of 70 sequenced BACs are within this FPC contig and their sequences were downloaded from GenBank on May 29, 2008. The physical map has been updated to the latest release (Maize golden path AGP v1, Release 4a.53). The detail about sequence annotation and gene prediction is illustrated in FIG. 7. A total of ˜1.5 Mb, comprising 44 unordered sequence fragments with 83 non-redundant predicted non-repetitive genes, were soft-masked for probe design. The uniqueness/repetitiveness of all the probes and physical locations of the probes were determined based on the collection of maize BAC sequences available March 2008. The array design was constructed by tiling at ˜5 bp spacing across the target regions. Probes with an average 15-mer frequency in the genome greater than 100 were excluded, as were probes that had greater than 5 close matches in the genome. A total of 41,555 probes were selected, and replicated at least 17 times on the array. To reconcile with the reference genome sequence, probes were remapped to B73 RefGen_v1 (Schnable, P. S. et al, Science, 326,1112-1115, (2009)). The final sequence interval was defined from the 1 kb upstream the most-left mapped probe (REGION0042FS000010140) to the 1 kb downstream the most-right mapped probe (REGION0028FS000002032), i.e. 183062553˜185609824 by on Chr. 3. Two fragments (183,315,664-183,553,126 by and 183,880,178-183,965,661 bp) were excluded for analyses because they were not present in the sequences used for probe design. This design may be ordered by requesting 081028_{—}Zea_{—}mays_schnable_cap.

The second array design was constructed by tiling at ˜15 bp spacing across 43 dispersed gene targets. Probes with an average 13-mer frequency in the genome greater than 500 were excluded, as were probes that had greater than 7 close matches in the genome. A total of 16,406 probes were selected and replicated 44 times on the array. This array comprises ˜350 Kbp of genomic space, but has only 123 Kb represented within the probes. This design may be ordered by requesting 080328_{—}maize_cap_springer_{—}1.

### Maize Sequence Capture and 454 Sequencing

DNA was isolated from 14-day-old seedlings of two maize inbreds, B73 and Mo17 using a reported protocol (Li, J. et al, Genetics 176, 1469-1482 (2007)). A 700 bp average insert size 454 GSFLX-Ti sequencing library was generated for each inbred and subjected to 7-cycle amplification using primers based upon the sequencing adapters. Amplicons were purified using a QIAquick/MinElute Spin Column (QIAGEN, Valencia, Calif.). The DNA concentration was determined using NanoDrop ND1000 (Thermo Scientific, Willmington, Del.) and the molecular weight range was determined using an Agilent Bioanalyzer2100 with a DNA7500 kit (Agilent Technologies, Santa Clara, Calif.). A total of 250 ng (or less) of each double stranded sequencing library was hybridized to the maize repeat subtraction at low stringency (37° C.) using the Mai Tai system (Scigene, Sunnyvale, Calif.) with 16 ul total NimbleGen hybridization cocktail solution along with a 20-fold molar excess of non-extendable primers complementary to the sequencing adapters. The rotation speed in the SciGene hybridization oven was set to setting 2. The hybridization cocktail was recovered by separating the two slides with the gasket array on the bottom (facing up) and the subtraction array (on the top, facing down). The remaining hybridization cocktail, containing the library fragments of interest (still on the gasket slide), was subjected to a second capture array aimed at the gene space of interest. The capture array was placed by inverting it (probes down) onto the hybridization cocktail on the gasket slide. The gasket slide remained in the Mai-Tai rig during the replacement. The capture array was then subjected to an additional 4 days of hybridization at 42.5° C. with the rotator set on setting 2. The capture array was washed as previously described (Albert, T. J. et al. Nat. Methods 4, 903-905 (2007)) and eluted non-selectively with a sodium hydroxide method available from Roche NimbleGen Inc. and summarized as follows:

12.5 ul of 10M NaOH was mixed with 987.5 ul of water to get a final concentration of 125 mM. The solution was vortexed well and spun down. Approximately 400 ul of the solution was added to the elution chamber and the chamber was returned to a horizontal position. Sample was incubated for 10 minutes. A pipette was used to mix by pulling liquid in and out of pipette tip 3 times and transferring to a clean 1.5 ml tube on the final mix when the liquid is in the pipette tip. Any residual liquid was removed with a small bore pipette tip and added to the 1.5 ml tube. Finally, Neutralization solution (16 ul of 20% Acetic Acid) was added and the eluted molecules were cleaned up with a Qiagen MinElute column.

The non-selectively eluted molecules were then amplified via the sequencing adapters (12 cycles) and the products were purified and quantified. The double stranded non-selectively eluted libraries were diluted for emPCR as recommended by 454 and sequenced using the 454 GSFLX-Titanium protocol under the manufacturer's conditions using a 4 or 16 region Titanium PTP. Prior to emPCR, the diluted double-stranded eluate libraries were heat treated at 95 deg C. for 2 minutes in a thermal cycler. This heating step was found to be essential to avoid amplification associated artifacts in the emPCR. The raw 454 capture reads with low quality (parameters: maximum average error=0.01, maximum error at ends=0.01) and short 454 reads (<200 bp) were removed using the LUCY program. (Chou, H. H. & Holmes, M. H., Bioinformatics, 17, 1093-1104 (2001))

### Data Analyses

To estimate on-target rates, all filtered B73 and Mo17 captured 454 reads were aligned to the B73 reference genome sequence, i.e., B73_RefGen_v1 (Schnable, P. S. et al, Science, 326, 1112-1115, (2009)) BLAST alignment criteria: 95% similarity and the total unaligned regions of both 5′ and 3′ ends of 454 reads <=15 bp). Sequence reads whose best match overlapped a target region were classified as on-target. For the probes that can be mapped outside Interval 377, target paralog region is defined as a non-redundant set of sequences of these probes that can be mapped both inside and outside Interval 377. Sequence reads with a best match overlapped with target paralog region are considered as on-paralog reads. Whole-genome CGH data was retrieved from NCBI GEO database (GSE16938) (Springer, et al. PLos Genetics, 5 (11), 2009). Only CGH probes within targeted regions were used to calculate normalized coverage. GFF files were generated for data visualization using NimbleScan (Version 2.4, NimbleGen). Shell and AWK scripts for the analysis pipeline are available upon request. Sequence alignments between B73 and Moll allelic sequences was conducted using VISTA (LAGAN alignment program used with default settings). CAP3 (Huang, X. & Madan, A., Genome Res. 9, 868-877, (1999)) was used for assembling Mo17 reads from the 43-Gene Array (parameters used: overlap percent identity>=95, overlap length>=50 bp).

### Results and Discussion

Over the past two decades several approaches to achieve a reduction in genomic complexity have been attempted, including EST sequencing, methyl-filtration, and high-Cot DNA selection (reviewed by Barbazuk et al., Bioessays 27, 839-848, (2005)). Each of these approaches has been successful in reducing genome complexity but none delivers sequences of interest in a targeted fashion as is possible with hybridization-based sequence capture. In initial experiments in which we utilized Cot1 DNA as a blacker we found that maize Cot1 DNA improved the performance of sequence capture relative to human Cot1 DNA (data not shown). Extending this idea would posit that adapting sequence capture technology for the many crop genomes would require the production of species-specific blocking agents for each of the many important crops. Published maize Cot1 production protocols have only ˜10% yield, making scaling production prohibitive from the perspective of genomic DNA consumption (Zwick, M. S. et al, Genome, 40, 138-142 (1997)). Further, in our hands, 16 out of 20 independent attempts at using the previously published Cot1-based protocol yielded fold enrichments that were at least an order of magnitude below those achieved in the current study (Schnable, Springer, Barbazuk and Jeddeloh, unpublished observation). We, therefore, investigated the use of a two-stage microarray sequence capture that might yield samples with consistently reduced complexity. A repeat-subtraction microarray was designed to remove DNA fragments that contain highly repetitive sequences.

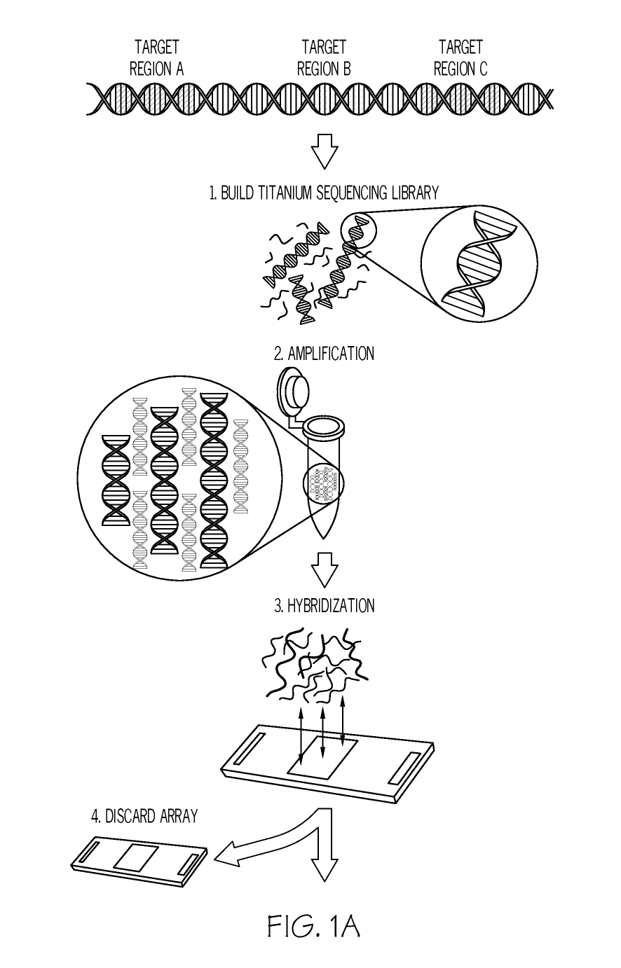

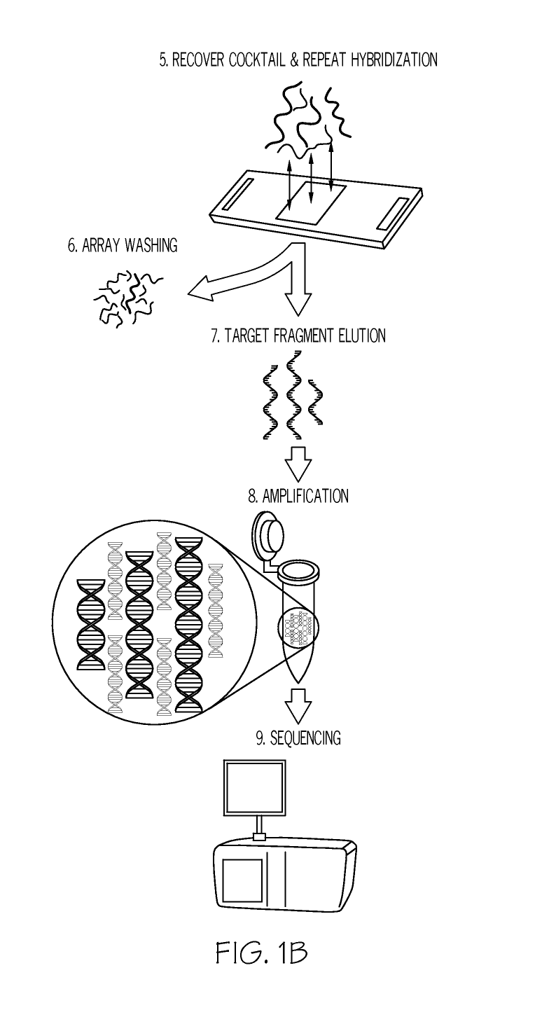

The process of array-based repeat subtraction sequence capture (RSSC) is depicted in FIG. 1. RSSC consists of two phases: reducing the abundance of repetitive sequences within the capture library and capturing target sequences from the resulting reduced complexity library. The publically available 454 GSFLX-Ti library construction protocol was utilized to produce a single-stranded A-B adapted sequencing library for either B73 or Mo17 inbreds with an average insert size of ˜700 bp. This library was then amplified via limited cycles of PCR using primers designed to the 454 Ti A/B adapters, purified, and quality checked. Next, RSSC was executed using a maize repeat array constructed by tiling probes across the maize accessions in a cereal repeat database. In addition to the maize repeat array, two specific capture arrays were designed. The first capture array (Interval 377 array) targets an ˜2.2 Mb genomic interval from Chromosome 3 of the B73 inbred. This array was designed based on the sequences of a series of 70 overlapping BACs. The Interval 377 array models situations in other crop genomes where a specific region of a sequenced genome is under investigation or where several sequenced BACs covering a region of interest are available from an otherwise unsequenced genome. One might expect this situation when chromosome walking in a large genome such as wheat or pine. The second capture array (43-Gene array) targets 43 genes dispersed throughout the genome. The 43-Gene array models the situation where several genes in an otherwise unsequenced genome are under investigation. For the Interval 377 array only, repeat sequences in the interval were masked prior to probe design (see Methods and Supplementary FIG. 1). Table 1 provides summary statistics about the design of both arrays.

TABLE 1

| | Interval 377 | |
|---|---|---|
| Array design statistics | Array^{a} | 43-Gene Array |
| Total length (bp) | 2,224,325 | 303,557 |
| Primary target space^{1, 2} after | 666,488 | No masking |
| repeat-masking (bp)^{b} | | |
| Length of target region (bp)^{c} | 277,305 | 280,749 |
| % of primary target space | 42% | 92% |
| covered by probes^{d} | | |
| Length of target paralogous | 45,434 | Not determined |
| region (bp)^{c} | | |
| No. non-TE protein-encoding | 40^{c} | 43 |
| genes | | |

^{a}Using the B73_RefV1 sequence as the reference sequence (Methods)
^{b}See Supplemental FIG. 1 for detailed method
^{c}The target region consists of a non-redundant set of sequences used for probe synthesis
^{d}Length of target region/Length of primary target space
^{e}Based on members of the “filtered gene set”⁶ that overlapped with the target region
Summary statistics for the maize capture data using two arrays and two genotypes are shown in Table 2.

TABLE 2

| | Interval 377 Array | | 43-Gene Array^{b} | |
|---|---|---|---|---|
| Genotype | B73^{a} | Mo17 | B73 | Mo17 |
| No. filtered reads^{c} | 268,350 | 132,162 | 16,135 | 30,367 |
| No. on-target reads^{d} (% of on- | 83,429 (31%) | 29,226 (22%) | 5,612 (35%), | 11,074 (36%) |
| target reads) | | | | |
| Fold enrichment^{e} | ~2,600 | ~1,800 | ~2,900 | ~3,000 |
| On-paralog reads^{f} (% of on- | 8,939 (3.3%) | 5,157 (3.9%) | ND^{g} | ND |
| paralog reads) | | | | |
| Fold enrichment for paralogs^{h} | ~1,700 | ~2,000 | ND | ND |
| Coverage | | | | |
| Percentage target bases | 98/97/94% | 82/78/70% | 91/73/20% | 81/70/46% |
| covered by ≧1/≧3≧10 capture | | | | |
| reads | | | | |
| Mean coverage of target bases | 106 | 38 | 6 | 12 |
| Mean coverage per 1,000 on- | 1.3 | 1.3 | 1.1 | 1.1 |
| target reads | | | | |

^{a}Two B73 regional captures were combined for calculation
^{b}Calculations were based on combined data from all genes
^{c}Reads remaining after removal of low-quality reads (Methods)
^{d}Reads mapping to a region overlapping with the target region
^{e}Percentage of on-target reads/(Length of target region/size of B73 reference[2.3Gb⁶])
^{f}The read mapped to a region overlapping with target paralog region
^{g}Not determined
^{h}Percentage of on-paralog reads/(Length of target paralogous region/size of B73 reference genome [2.3Gb⁶])

Finally, SNP prediction using reads captured from B73 and Mo17 is shown in Table 3.

TABLE 3

| | | | | No. genes |
|---|---|---|---|---|
| | | No. | No. high- | with high- |
| | Input data^{a} | SNP | quality SNPs^{b} | quality SNPs |
| Interval 377 | B73-all | 8,531 | 98 | 2 |
| | B73-target^{b} | 23 | 5 | 1 |
| | Mo17-all | 8,044 | 1,693 | 35 |
| | Mo17-target | 1,649 | 1,357 | 34 |
| 43-Gene Set | B73-all | 170 | 31 | 11 |
| | B73-target | 144 | 30 | 11 |
| | Mo17-all | 2,249 | 1,240 | 40 |
| | Mo17-target | 1,790 | 1,221 | 39 |

^{a}Two sets of B73 and Mo17-derived sequence reads were used for SNP prediction: all filtered reads (“all”) and on-target reads (“target”).
^{b}High-quality SNPs are those that are mono-allelic in all aligning reads. In addition, SNPs identified within repetitive DNA regions of Interval 377 were removed (Methods).

### Broader Applicability of RSSC

Use of the described protocol achieved ˜1,800-3,000-fold enrichment of both a defined chromosomal interval and a set of dispersed genes. This enrichment is comparable to that achieved from the human genome (Albert, T. J. et al, Nat. Methods 4, 903-905 (2007)). For both captures 80-98% of targeted bases were covered by captured sequences. The mean coverage of the target regions per 1,000 on-target reads are similar for captures from the two different arrays (1.3 vs. 1.1), highlighting the overall robustness of the approach. Therefore, the RSSC protocol provides a method to resequence targeted genomic regions of the maize genome, and it is expected to exhibit similar levels of performance in other genomes. The ability to design reagents required for repeat subtraction in silico significantly reduces the technical hurdles of applying sequence capture across diverse species. Because highly repetitive elements can be discovered using only limited amounts of whole genome shotgun sequencing data, in combination with next generation sequencing technologies it is feasible to design species-specific repeat-subtraction arrays with limited investment of resources. Hence, the present RSSC protocols can be applied not only to species with sequenced reference genomes, but also to those whose genomes have not yet been sequenced. Importantly, polymorphism analyses conducted in the absence of a fully sequenced reference genome will not be substantially cumbersome. This technology can be applied for studies of population genetics, cloning of loci controlling quantitative variation and allele mining in crops, model organisms and importantly, non-model species.

### Example 2

### Solution-Based Repeat Subtraction-Mediated Sequence Capture (RSSC) for Maize

### Repeat Subtraction Array

A custom NimbleGen 3×720K sequence Capture microarray was synthesized to contain maize repetitive elements in the MAGI Cereal Repeat Database (v3.1; http://magi.plantgenomics.iastate.edu/repeatdb.html) and the Maize Repeat Database (v 4; http://maize.jcvi.org/repeat_db.shtml). Each probe contained 15mer sequence on both the 5 and 3 prime end to facilitate amplification with Insitu primers. There are 2.1M total probes on the array, though only the center subarray containing the 720K probes was utilized.

### Maize NimbleGen Sequence Capture Array Design

The array design was the same as in example 1.

### Maize Sequence Capture Library

DNA was isolated from 14-day-old seedlings of inbred line B73 using reported protocol (Li et al. 2007). A 700 bp average insert size 454 GS FLX-Titanium sequencing library was generated and subjected to 8 cycles of amplification using primers based upon the sequencing adaptors. Amplicons were purified using Qiagen MinElute Column and quantified using the NanoDrop ND1000.

### Probe Pool and Repeat Subtraction

Solution phase repeat subtraction array was overlaid with a gasket array from Grace Bio-Labs (Bend, Oreg.) and subjected to 30 cycles of PCR, on the array surface, to produce repeat probe pools In situ as described in WO2009053039, Albert and Rodesch: Methods and System for the Solution Based Sequence Enrichment and Analysis of Genomic Regions and incorporated in total herein. The In situ PCR product was cleaned using an Qiagen Qiaquick column and eluted in water. The sample was quantified using the NanoDrop ND1000 and diluted to a concentration of 25 ng/μl. This diluted probe pool was then used as template for asymmetric PCR. Asymmetric PCR used one primer, labeled with biotin, in excess to force the amplification of only one strand of the double stranded DNA. The biotin labeled primers allowed for the removal of the probe repetitive elements hybridization complex by binding the biotin to Streptavidin beads (Invitrogen, Inc. (Carlsbad, Calif.)). Fifteen cycles of asymmetric PCR was done for forward and reverse strands to generate probe pools, respectively as described in WO2009053039. Forward and reverse strands were quantified using the NanoDrop ND1000 and 100 ng of each probes were combined into one 1.5 ml. In a separate tube 500 ng of maize Titanium Library was added along with a 100 fold molar excess of non-extendable primers complementary to the sequencer adapters. Both tubes were dried down in an Eppendorf Vacufuge (Hauppauge, N.Y.) at 60° C. for 10 minutes. To rehydrate probes 4.8 μl of water was added and tube was placed into a heating block at 70° C. for 10 minutes. Concurrently, 8.0 μl of Hybridization buffer and 3.2 μl of Component A were added to the sample and placed in a heating block at 95° C. for 10 minutes. Post incubation both tubes were vortexed and spun down. DNA library in hybridization buffer and component A were added to the probe pool, mixed using a pipette tip, then transferred to a 0.2 ml PCR tube using the same pipette tip. The probe pool, DNA, and non-extendable primers were placed into a thermocycler at 95° C. for 2 minutes, to ensure complete denaturation of the test DNA, followed by incubation at 37° C. for 8-24 hours.

To bind repetitive elements the sample needed to be incubated with Streptavidin beads. This process bound the biotin labeled probes, that were hybridized to the repetitive DNA, allowing for the removal or said elements. First, 100 μl of beads were transferred to a 1.5 ml tube and pelleted against the tube using a magnetic particle collector (MPC) (Invitrogen, lnc., Carlsbad, Calif.) and all liquid was removed. Beads were washed two times with a bead binding and wash buffer consisting of the following: 10 μl 1 molar TRIS-HCl, 2 μl of 0.5 molar EDTA, 400 μl of 5 molar NaCl, and 588 ul? of sterile water. After the second wash beads were pelleted against the tube wall with the MPC and all buffer was removed. Incubated sample was added to the tube containing the beads and lightly vortexed and spun down to re-suspend beads into sample solution. The biotin was bound to the Streptavidin beads by incubating tube in a thermocycler at 47° C. for 45 minutes. Sample was mixed at 15 minutes intervals with a pipette tip to prevent beads from settling. Following the incubation sample was place back into MPC to pellet the beads contain the biotin labeled probes and repetitive DNA elements complex. Aqueous repeat free DNA was then removed from tube contain bound beads and placed into a clean 1.5 ml tube. Volume of sample was measured and brought to 16 μl with the following mixture: 4.8 μl water, 8 μl hybridization buffer, and 3.2 μl Component A. Sample was then subject to the standard sequence capture work flow as described in the solid phase repeat subtraction.

Sequencing results are shown in table 4 below:

TABLE 4

| Sample | B73 | B73 | B73 |
|---|---|---|---|
| Protocol | SPRS | SPRS | Cot-1 Blocker |
| Array | 5873 | 5874 | 5258 |
| Total Reads | 29993 | 27391 | 57032 |
| Percent Reads Uniquely Mapped | 89.1 | 85.2 | 75.7 |
| Percent Basepairs HSP Trimmed | 639 | 5.8 | 5.4 |
| Percent target Bases Covered | 97.6 | 77.9 | 43.5 |
| Percent Reads in Target Region | 23.3 | 3.8 | 2 |
| Average Coverage | 15 | 2.1 | 1.1 |
| Median Coverage | 14 | 2 | 0 |

### Example 3

### Solution-Based Repeat Subtraction-Mediated Sequence Capture (RSSC) for Canola

### Repeat Subtraction Array

Complete BAC sequences from Brassica rapa subsp pekinensis were downloaded from GenBank in April 2009. A total of 970 BAC sequences were collected, representing 125.4 Mbp of the Brassica genome. The RepeatScout application suite (v1.0.5) was used to define a set of repeat sequences. Briefly, the build_Imer_table application was used to build a table of frequencies, using the default settings for the application. Then the RepeatScout application, with the frequency table, was used to create a set of 12316 repeat sequences, totaling 10.2 Mbp. The repeat sequences ranged in size from 50 by to 15670 bp, with an average size of 829 by and a median size of 236 bp. Sequence capture probes were then generated for these repeat sequences by tiling. Additional probes were generated by tiling through 117 Mbp of whole genome shotgun (WGS) sequencing reads from canola. A 13-mer frequency histogram was generated from the Brassica BAC sequences described above and used to calculate the average 13-mer frequency found in each probe. Probes with an average 13-mer frequency greater than a specified threshold were classified as repetitive. The non-redundant set of repetitive probe sequences was then used on the array design. For the solid phase design a 50 bp tiling interval was used on the set of repeat sequences, and a 100 bp tiling interval on the WGS sequence. A threshold of 100 was used to classify the probes from the WGS sequence as repetitive. The probes were placed on the array in both forward and reverse orientation. There were a total of 296642 (2×148321) probes from the repeat sequence set and 420018 (2×210009) probes from the WGS sequence. For the solution phase design a 25 bp tiling interval was used on the set of repeat sequences, and a 50 bp tiling interval on the WGS sequence. A threshold of 80 was used to classify the probes from the WGS sequence as repetitive. The probes were placed on the array the forward orientation only. There were a total of 287813 probes from the repeat sequence set and 424804 probes from the WGS sequence.

### Canola NimbleGen Sequence Capture Array Design

A total of 769 Canola EST sequences were used as the target sequences, totaling 514 kb. Sequence capture probes were generated at a 1 bp tiling interval, ranging in size from 59 to 97 bp. A total of 90000 probes were selected to represent the EST sequences, and these probes were replicated 8 times on the array design.

The work flow for canola was identical to maize except for the following: Specific repeat subtraction array and sequence capture arrays were design from the canola genome. Sequence capture was preformed with 100 ng of Titanium Library in canola, while 500 was used in 500 ng in maize. All other process were identical to that described above in the Maize description and the Roche NimbleGen users guide.

The sequencing results are shown in Table 5 below:

TABLE 5

| | Design | | | | | | | |
|---|---|---|---|---|---|---|---|---|
| | EST | EST | EST | EST | EST | EST | EST | EST |
| Sample | Av_4462 | Av_4463 | Av_4406 | Av_4444 | Mo_4445 | Mo_4508 | Mo_4475 | Mo_4476 |
| Total Reads | 59501 | 58386 | 87056 | 63874 | 78467 | 70515 | 64651 | 59853 |
| Percent Reads | 14.10% | 22.00% | 34.70% | 27.70% | 35.90% | 30.80% | 36.90% | 11.60% |
| Uniquely Mapped | | | | | | | | |
| Percent Basepairs HSP | 8.80% | 14.70% | 22.90% | 18.70% | 22.90% | 20.10% | 24.00% | 7.00% |
| Trimmed | | | | | | | | |
| Percent target Bases | 81.4 | 89.1 | 96.8 | 94.6 | 97.1 | 95.5 | 95.9 | 71.2 |
| Covered | | | | | | | | |
| Percent Reads in | 73.7 | 80.4 | 84.5 | 82 | 84.3 | 83.6 | 85.9 | 65.3 |
| Target Region | | | | | | | | |
| Average Coverage | 2.4 | 3.9 | 10.3 | 5.8 | 9.1 | 7.1 | 8 | 1.8 |
| Median Coverage | 2 | 3 | 9 | 5 | 8 | 6 | 7 | 1 |

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

1. A method of enriching for target nucleic acid sequences in a sample, the method comprising:
a) applying a sample comprising nucleic acid sequences, wherein said nucleic acid sequences comprise non-target and target nucleic acid sequences, to a first set of hybridization probes wherein said hybridization probes comprise sequences complementary to the non-target nucleic acid sequences in the sample, to allow hybridization,
b) separating a solution comprising non-hybridized target nucleic acid sequences from the hybridized non-target sequences,
c) applying the solution comprising non-hybridized target nucleic acid sequences to a second set of hybridization probes wherein said second set of hybridization probes comprise sequences complementary to the target nucleic acid sequences to allow hybridization, and
d) eluting said hybridized target nucleic acid sequences from the second set of hybridization probes thereby enriching for target nucleic acid sequences in a sample.

2. The method of claim 1 in which the non-target nucleic acid sequences are repetitive sequences.

3. The method of claim 1 in which the target and non-target nucleic acid sequences are mRNA, cDNA or rRNA sequences.

4. The method of claim 1 in which the target and non-target nucleic acid sequences are plant nucleic acid sequences.

5. The method of claim 4, wherein said plant nucleic acid sequences are from maize or canola.

6. The method of claim 1 in which steps a) and c) take place on a solid phase.

7. The method of claim 6 in which the solid phase is a microarray.

8. The method of claim 1 in which at least one of the steps a) and c) takes place in solution.

9. A method of enriching for target nucleic acid sequences in a sample comprised of target and non-target nucleic acids, the method comprising:
a) generating a first set of hybridization probes comprising sequences complementary to non-target nucleic acid sequences;
b) generating a second set of hybridization probes comprising sequences complementary to target nucleic acid sequences;
c) combining the first set of probes with the sample to allow the first set of probes to hybridize to non-target nucleic acids;
d) removing the hybridized first set of probes from the sample to form a first enriched solution comprising the target nucleic acid sequences;
e) combining the second set of probes with the first enriched solution to allow the second set of probes to hybridize to target nucleic acids;
f) removing the hybridized second set of probes; and
g) eluting the target sequences from the hybridized second set of probes to form a second enriched solution comprising the target nucleic acid sequences.

10. The method of claim 9 in which step c) takes place on a microarray.

11. The method of claim 9 in which the first set of hybridization probes is generated in solution in step a) and the hybridization step c) takes place in solution.

12. The method of claim 11 in which a microarray is used to generate the first set of hybridization probes in solution in step a).

13. The method of claim 12 in which the first set of hybridization probes is generated in solution from said microarray in step a) by means of a first polymerase chain reaction.

14. The method of claim 13 in which the first set of hybridization probes generated in solution by means of a first polymerase chain reaction in step a) is further amplified by means of a second polymerase chain reaction.

15. The method of claim 14 in which the second polymerase chain reaction is asymmetric.

16. The method of claim 15 further comprising introduction of a specific binding pair member in the asymmetric polymerase chain reaction.

17. The method of claim 16 in which the specific binding pair member is a biotin label derived from a biotin labelled primer.

18. The method of claim 17 in which the biotin-labelled hybridization probes are paired with a streptavidin solid phase as a means of separation of solutions containing unhybridized nucleic acids from the said first set of hybridization probes.

19. The method of claim 9 in which the second set of hybridization probes in step b) is generated on a microarray and step e) takes place on said microarray.

20. The method of claim 9 in which the second set of hybridization probes in step b) is generated in solution and step e) takes place in solution.

21. The method or claim 20 in which a microarray is used to generate the second set of hybridization probes in solution in step b).

22. The method of claim 21 which the second set of hybridization probes in step b) is generated in solution from said microarray by means of a first polymerase chain reaction.

23. The method of claim 22 in which the second set of hybridization probes in step b) generated in solution by means of a first polymerase chain reaction is further amplified by means of an second polymerase chain reaction.

24. The method of claim 23 in which the second polymerase chain reaction is asymmetric.

25. The method of claim 24 further comprising introduction of a specific binding pair member to the amplified hybridization probes in the asymmetric polymerase chain reaction.

26. The method of claim 25 in which the specific binding pair member on the amplified hybridization probes is a biotin label derived from a biotin labelled primer.

27. The method of claim 26 in which the biotin-labelled hybridization probes are paired with a streptavidin solid phase as a means of separation of solutions containing unhybridized nucleic acids from the said first or second set of probes.

28. A method of enriching for target nucleic acid sequences in a sample comprised of target and non-target nucleic acids, the method comprising:
a) applying a sample to a substrate comprising hybridization probes wherein said probes comprise sequences complementary to non-target nucleic acid sequences and sequences complementary to target nucleic acid sequences, and wherein said sequences complementary to non-target nucleic acid sequences and sequences complementary to target nucleic acid sequences are separately located to allow hybridization of the sample to the probes, and
b) selectively eluting the hybridized target nucleic acid sequences from the probes thereby enriching for target nucleic acid sequences in a sample.

29. The method of claim 27, wherein said probes are sequences derived from plant nucleic acids.

30. The method of claim 28, wherein said plant nucleic acids are maize or canola nucleic acids.
